(19) United States
(12) Patent Application Publication
(10) Pub. No.: US 2018/0038825 A1
(43) Pub. Date: Feb. 8, 2018

(54) Multiple Sensor System for Breath Acetone Monitoring

(71) Applicant: Breathometer, Inc., Burlingame, CA (US)

(72) Inventors: Timothy Ratto, San Mateo, CA (US); Royal Wang, Dublin, CA (US); Jonathan Gallagher, San Francisco, CA (US); Elllery Wong, San Mateo, CA (US); Likang Xue, Santa Clara, CA (US); Kenton Ngo, San Francisco, CA (US); Jeffrey Zalewski, Corte Madera, CA (US); Robert Edwards, Menlo Park, CA (US)

(21) Appl. No.: 15/226,605

(22) Filed: Aug. 2, 2016

(51) Int. Cl. G01N 27/416; G01N 27/407; G01N 33/497

(52) U.S. Cl. CPC G01N 27/4163; G01N 33/497; G01N 27/407; G01N 2033/4975

(57) ABSTRACT

A multiple sensor array for breath acetone measurement has a semiconductor sensor that measures concentration of a set of volatile organic compounds. An electrochemical sensor measures volatile organic compounds or other gasses such as carbon monoxide. At least one correction sensor that measures other breath properties can also form a part of the multiple sensor array, providing information on physical properties such as pressure and temperature, and information related to individual gases including carbon monoxide, water vapor, hydrogen, ethylene oxide, and the like. An acetone concentration calculation module takes measured values from the multiple sensor array to measure breath acetone, which can be stored and displayed for a user.

### TECHNICAL FIELD

The present disclosure relates generally to a multiple sensor system for breath acetone monitoring. In certain embodiments a smartphone or other computing device can be used for processing sensor measurements and providing a visual display of results.

### BACKGROUND

Quantitative or semi-quantitative measurement of breath ketones such as acetone has long been available in research, laboratory, or hospital settings. These measurements can allow for determination of abnormal health conditions such as diabetes, or track metabolic rate as a function of ketone production. Levels of produced breath acetone can also reflect rates of lipid oxidation (i.e. fat burning), making it a desirable tool for monitoring diet efficacy. Typically, expensive instrumentation including gas chromatographs, mass spectrometers, reactive ion spectrometers, or ion flow tube mass spectrometers are used for limited duration test trials. These instruments are not generally suitable for personal or home use, and require skilled operators and frequent calibration.

Unfortunately, breath acetone can be difficult to consistently measure. In healthy individuals, breath acetone is typically present at the level of only a few hundred parts per billion to tens of parts per million. Further, the composition of acetone can be difficult to distinguish from other volatile organic compounds (VOCs) in breath, of which hundreds of detectible compounds exist. Semiconductor or electrochemical sensors typically do not have the required selectivity or sensitivity to acetone, and may require pretreatment or filtering of exhaled breath to remove interfering VOCs. Other problems for home use relate to cost, requirement of skilled operators, sensor drift, and calibration, all of which can make accurate determination of breath acetone by a home user difficult or impossible.

### SUMMARY

A breath acetone measurement system can include a multiple sensor array having a first semiconductor sensor that measures a concentration of a set of volatile organic compounds. A second semiconductor sensor measures concentration of a subset of the volatile organic compounds measured by the first semiconductor sensor, and a third electrochemical sensor measures a further subset of volatile organic compounds measured by the first and second semiconductor sensors. At least one correction sensor that measures other breath properties can also form a part of the multiple sensor array, providing information on physical properties such as pressure and temperature, and information related to minor gases including carbon monoxide, water vapor, and the like. An acetone concentration calculation module takes measured values from the multiple sensor array to measure breath acetone.

In another embodiment, a multiple sensor array has a semiconductor sensor that measures concentration of a set of volatile organic compounds. An electrochemical sensor measures a further subset of volatile organic compounds measured by the semiconductor sensor. At least one correction sensor that measures other breath properties can also form a part of the multiple sensor array, providing information on physical properties such as pressure and temperature, and information related to minor gases including carbon monoxide, water vapor, hydrogen, ethylene oxide, and the like. An acetone concentration calculation module takes measured values from the multiple sensor array to measure breath acetone. In one embodiment, a smartphone, tablet, or other personal computing device provides computational support for the acetone concentration calculation module, as well as providing a visual display of results.

In another embodiment, a method for determining breath acetone includes the steps of providing a multiple sensor array for measuring concentration of a set of volatile organic compounds using a first semiconductor sensor. Optionally a subset of volatile organic compounds detected by the first semiconductor sensor can be measured using a second semiconductor sensor. A further subset of volatile organic compounds can be measured using a third electrochemical sensor, while other breath properties can be measured using at least one correction sensor. These sensor measurements can be sent to a calculation module that determines breath acetone concentration using the measured.

### BRIEF DESCRIPTION OF THE DRAWINGS

FIG. 1 is an illustration of a breath acetone measurement system including a multiple sensor array;

FIG. 2 is flow chart illustrating steps in use of a breath acetone measurement system;

FIG. 3A is an illustration of a breath acetone measurement system that includes a multiple sensor array and a connected smartphone for data processing;

FIG. 3B is an illustration of a breath acetone measurement system with integrated processing and display capability;

FIG. 4A and FIG. 4B are devices schematic illustrating one embodiment of a breath acetone measurement system;

FIGS. 5A, 5B, and 5C are graphs illustrating sensor properties;

FIG. 6 is a graph illustrating representative acetone readings and their metabolic significance; and

FIG. 7 is a graph illustrating daily metabolic rate and five selected times when breath acetone measurements are completed.

### DETAILED DESCRIPTION

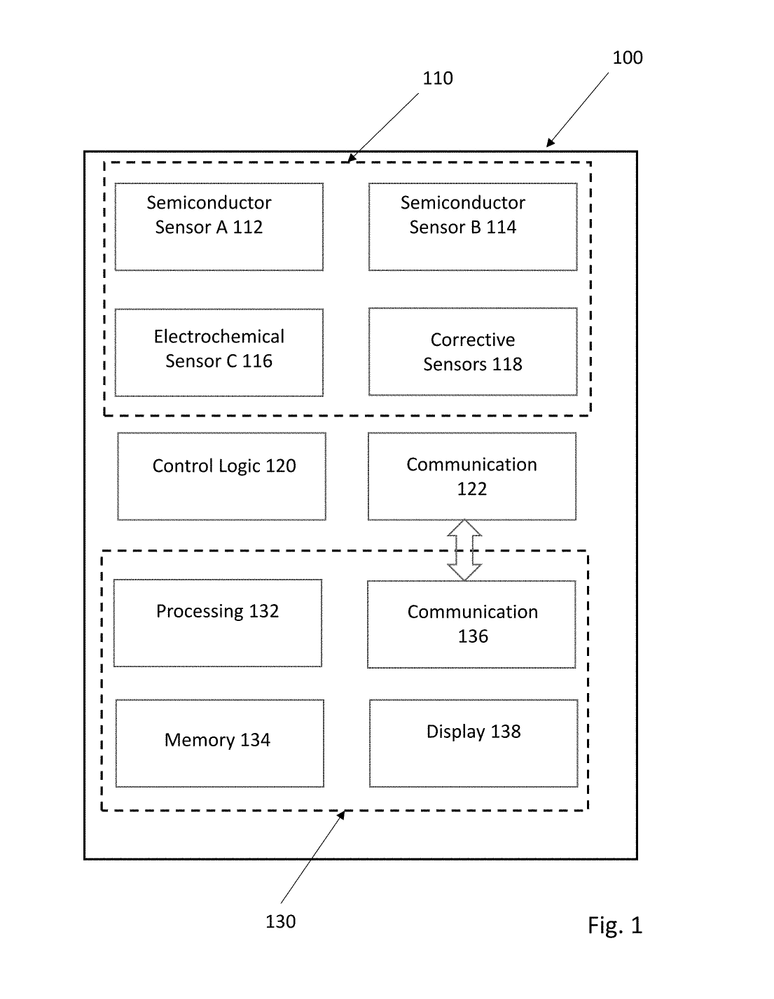

FIG. 1 is diagram showing elements of a system 100, including a multiple sensor array 110 suitable for breath acetone measurement according to one embodiment. The multiple sensor system uses a combination of semiconductor based, non-selective volatile organic compounds (VOC) sensors 112 and 114, along with an electrochemical sensor 116 (ECS). Additional corrective sensors 118 to detect various chemical (e.g. water vapor) or physical (pressure and temperature) characteristics can also be used to provide necessary correction factors.

In one embodiment, the semiconductor sensor “A” 112 broadly detects a range of VOC types. A second semiconductor “B” 114 detects a narrower range of VOC types than sensor “A”. A third electrochemical sensor “C” 116 is used to detect selected gasses (for example an even narrower range of VOC's or other gasses such as CO or H). The third or additional electrochemical sensors can also be used to detect a other gases, including but not limited to a further subset of volatile organic compounds measured by the semiconductor sensors. For example, hydrogen or ethylene oxide can be measured with one or more electrochemical sensors. Data from these three sensors, along with corrective data from other sensor types, is used to determine breath acetone at parts per billion (ppb) to parts per million (ppm) levels. Preferred detection levels are 100-20000 ppb, particularly 200-5000 ppb. In one embodiment, sensor “C” 116 can be a three electrode electrochemical sensor commonly used to detect ethylene oxide.

Sensor operation is controlled by a control logic module 120 that turns on or off, calibrates, and otherwise manages the sensor array 110. Data from sensor array 110 is transferred for storage or further processing using communication module 122. In integrated embodiments, module 122 is identical to module 136, and data is processed (132), stored (134), and displayed (138) locally. In other embodiments, communication module 136 provides input to a separate computing device such as a smartphone, table, laptop or computer 130 having a separate processing, memory, and display system. Interaction can be provided by wireless or wired network interface. Input can be through a touchpad, by voice control, or by typing. The display can be a conventional OLED or LCD, or other suitable display. In some embodiments, audio feedback can be provided instead or in addition to visual display. Typically, a user interface is accessible by the user through a smartphone or tablet application such as are provided for Android™ or iPhone™ applications.

Optionally, data and control signals can be received, generated, or transported between varieties of external data sources, including wireless networks or personal area networks, cellular networks, or internet or cloud mediated data sources. In addition, local data storage (e.g. a hard drive, solid state drive, flash memory, or SRAM) that can allow for data storage of user-specified preferences or protocols. In one possible embodiment, multiple communication systems can be provided. For example, system 100 can be provided with a direct WiFi connection (802.11b/g/n), as well as a separate 4G cell connection provided as a back-up communication channel.

Because data is health related and may be personally sensitive, system 100 usage can require secure identification of a user through possession of a designated device, through passwords or biometric authentication, or by other suitable enrollment and authentication procedures. Typically, a user will have identifying password that is used in conjunction with a password protected smartphone, tablet, or computer.

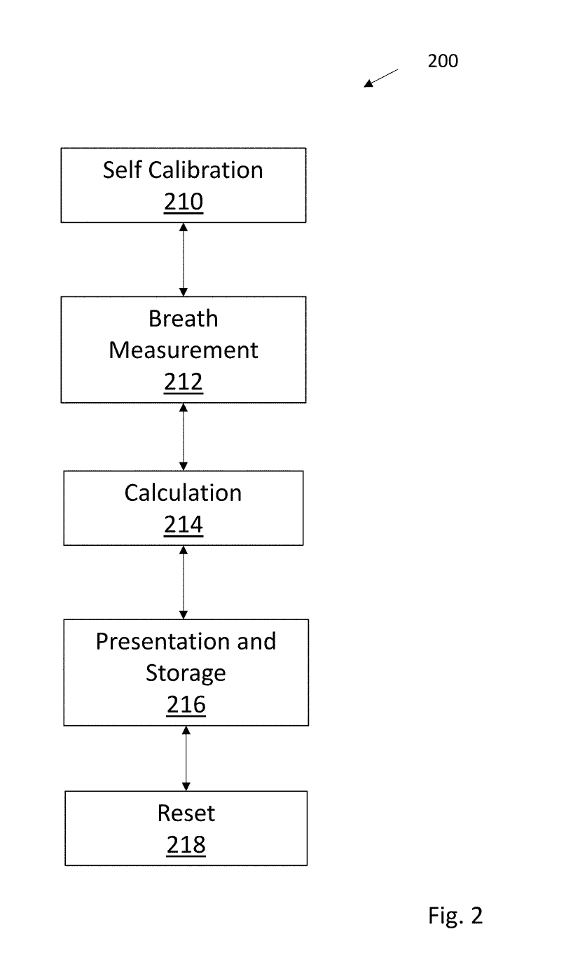

FIG. 2 is flow chart 200 illustrating steps in use of a breath acetone measurement system. After an initial self calibration step 210, breath measurement is taken using a device suited to accommodating at least five to ten seconds of user breath flow. As the breath flows across the multiple sensors, readings are taken for non-selective volatile organic compounds (VOC) sensors, along with one or more electrochemical sensors (ECS) to detect hydrogen or selected VOC subsets. Additional readings are taken by corrective sensors to detect various chemical (e.g. water vapor and carbon monoxide) or physical (pressure and temperature) characteristics. The data is transferred to a calculation unit that weights individual sensor readings (step 214), and provides a single measurement of breath acetone for an identified person at a determined date/time. This data can be immediately presented, or stored for later usage and presentation (step 216). Additional measurements can require a reset (step 218) that involves heating selected sensors to drive off VOCs and prepare for next usage.

Advantageously, use of non-specific sensors in the described manner eliminates the need for expensive acetone specific sensors, or complex filtering or capture techniques to remove non-acetone VOCs before measurement. As compared to enzyme, colorimetric, or other conventional acetone assay methods, the described acetone sensor system is reusable, requiring only a several minute self-cleaning cycle to heat and burn off VOCs from sensor surfaces before being ready to use again.

A class of non-specific, semiconductor sensors known as metal oxide sensors are useful in one embodiment. A metal oxide sensor includes both a metal-oxide sensing layer and a heater. Resistance of the metal oxide sensing layer is altered when target gasses are present. This type of sensor is relatively nonselective for many types of gasses. In operation, oxidizing gases such as nitrogen dioxide and ozone cause resistance to increase, while for reducing gases like VOCs and carbon monoxide, the resistance goes down. Regulating the heater power and/or doping the metal oxide layer can be used to roughly adjust the selectivity of the sensors, however all known metal oxide sensors show some reactivity to a variety of gasses. For breath detection, metal oxide sensors that show the highest sensitivity to reducing gasses are preferred. This typically means sensors with tin oxide, with and without dopants such as tungsten, palladium, platinum, titanium, lanthanum, zinc and other dopants, heated to temperatures between 300-700° C., preferably 400° C. Because higher heater temperatures increase a set of analytes that can be detected by a specific sensor type such as tin oxide, a second identical sensor type can be heated to a lower temperature of between 25-300° C. (preferably about 125° C. to 175° C.) enabling a reduction in reactivity, and consequent sensitivity to a subset of analytes that are detected by the sensor. Alternately, in another embodiment, sensors that have different dopants can be used. For example, a tin oxide sensor and a tungsten-doped tin oxide sensor with or without different heater temperatures, can be used to vary selectivity to a subset of analytes.

In order to properly detect acetone in breath using metal oxide sensors, one of the metal oxide sensors should be able to detect acetone at concentrations ranging from 250-25,000 ppb, preferably between 500-5000 ppb. In addition to acetone, other breath analytes can also be detected by metal oxide sensors, particularly carbon monoxide, hydrogen, hydrocarbons and others. For metal oxide sensors that are cross-reactive with these analytes, the sensors should be able to detect CO in the range from 1-20 ppm, hydrogen from 2-20 ppm and hydrocarbons from 100-1000 ppb. For other cross-reactive analytes the sensors should be able to detect the analytes in the concentration ranges typically found in human breath. Metal oxide sensors are also sensitive to changes in relative humidity, for breath applications the resistance of the metal oxide sensors should not change more than 60% upon a change in RH of 60%.

In one example embodiment, two identical tin-oxide sensors can be used. One is run at a heater temperature above 300° C., preferably 400° C. reactive to acetone, CO, hydrogen and hydrocarbons, and the other tin-oxide sensor run at a heater temperature below 300° C., preferably 150° C., reactive to a subset of the above analytes such as CO, hydrogen and hydrocarbons. Subtracting the two sensor outputs gives a result that is correlated with the amount of acetone in breath. To increase accuracy, the metal oxide sensor outputs can be corrected for humidity and/or differences in sensitivities to CO and hydrogen. The use of a relative humidity/temperature sensor and electrochemical sensors selective for CO in the range of 1-20 ppm and/or hydrogen in the range of 2-20 ppm allow for correction in one embodiment.

In another embodiment, one tungsten-doped tin-oxide sensor and one undoped tin-oxide sensor can both be run at identical heater temperatures below 300° C. Subtraction of the sensor outputs can correlated with breath acetone. Additional humidity and temperature, and CO and hydrogen correction result in a breath acetone measurement.

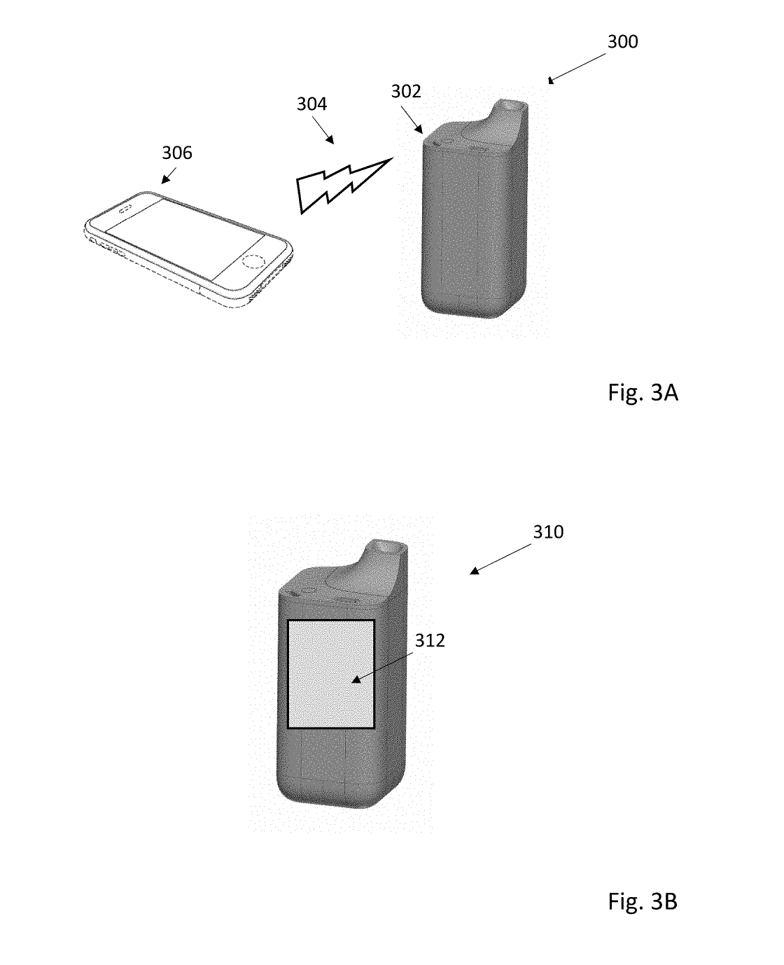

FIG. 3A is an illustration of a breath acetone measurement system 300 that includes a multiple sensor array inside case 302 and a connected smartphone 306 for data processing and presentation. Breath characteristics are determined by a multiple sensor system uses a combination of semiconductor based, non-selective volatile organic compounds (VOC) sensors based on metal oxide sensors heatable to predetermined temperatures. Additional electrochemical or other corrective sensors can to detect various chemicals (e.g. selected VOCs, hydrogen, water vapor and carbon monoxide) or physical (pressure and temperature) characteristics. The raw data can be transferred by wireless or wired communication 304 to a smartphone. A smartphone application can be used to process raw data, make corrections, display, and store data. In other embodiments, some or all of the data can be transferred to laptops, personal computers, servers. cloud servers and the like for additional processing or storage.

FIG. 3B is an illustration of a breath acetone measurement system 310 with integrated processing and communication capability. Similar to that discussed in connection with FIG. 3A. In this embodiment however, a smartphone is not necessary for processing and display. The system 310 can process, display, and store data of interest to a user. As will be appreciated, wireless or wired communication to transfer data to smartphones, laptops, personal computers, servers, cloud servers and the like is still possible.

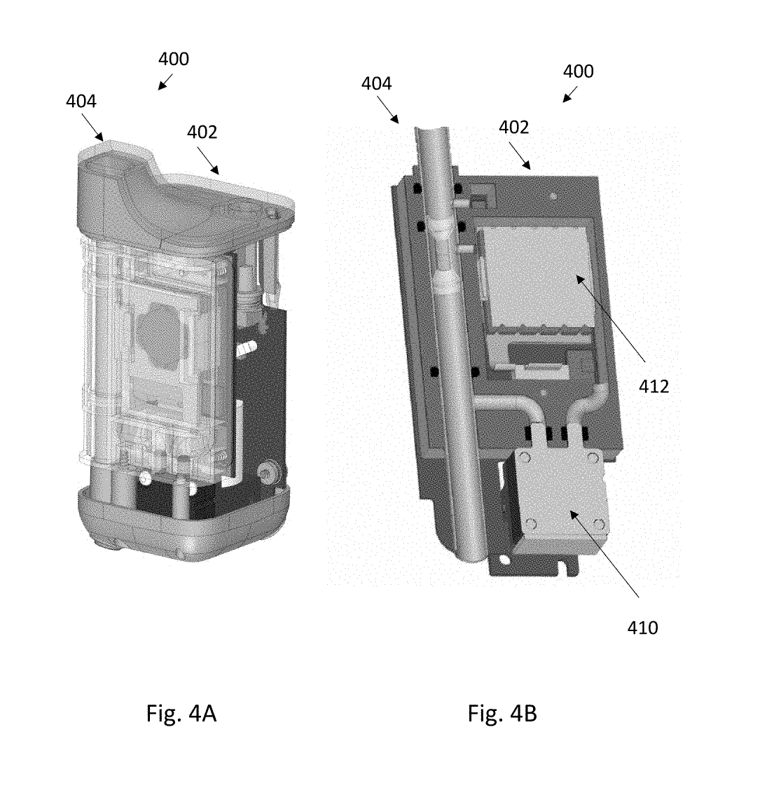

FIG. 4 is a device schematic illustrating one embodiment of a breath acetone measurement system 400. As seen in partial cutaway view, a system 400 can include a case 402 that holds a breath tube 404. A user breathing into breath tube 404 provides a breath sample to be measured. A pump 410 connected to the breath tube causes the breath sample to pass through a multiple sensor system 412, which in turn has data locally read and preliminarily processed by processing unit.

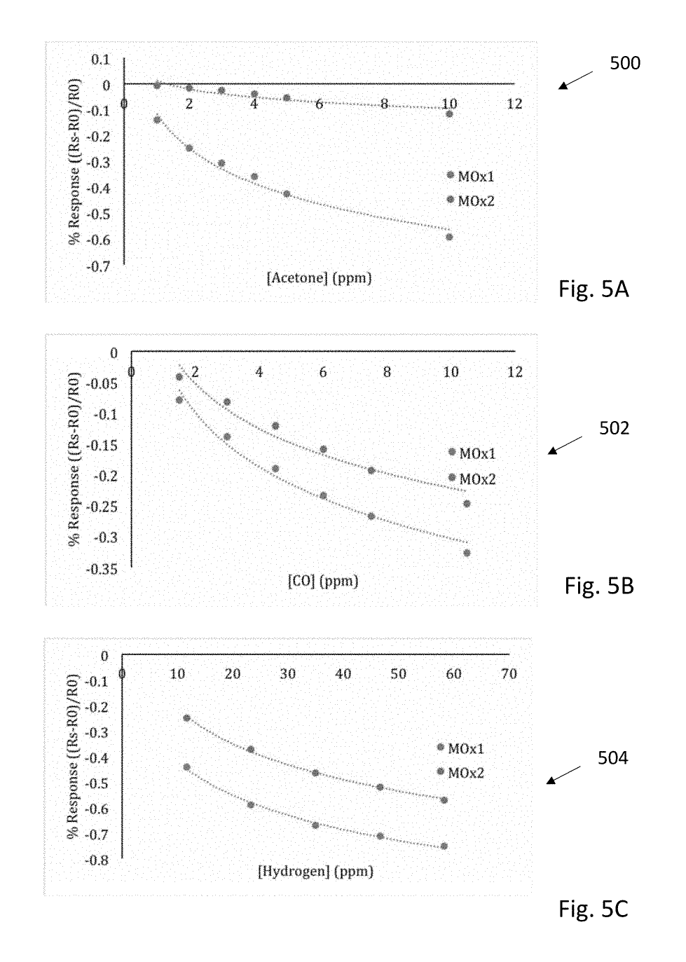

FIGS. 5A, 5B, and 5C are graphs illustrating sensor properties. Graph 500 illustrates respective response curves for two metal oxide sensors run at different temperatures and configured to detect acetone. Graph 502 illustrates respective response curves for two metal oxide sensors run at different temperatures and configured to detect carbon monoxide (CO). Graph 504 illustrates respective response curves for two metal oxide sensors run at different temperatures and configured to detect hydrogen. Collectively, this response data (along with various correction factors) can be used to calculate breath acetone levels.

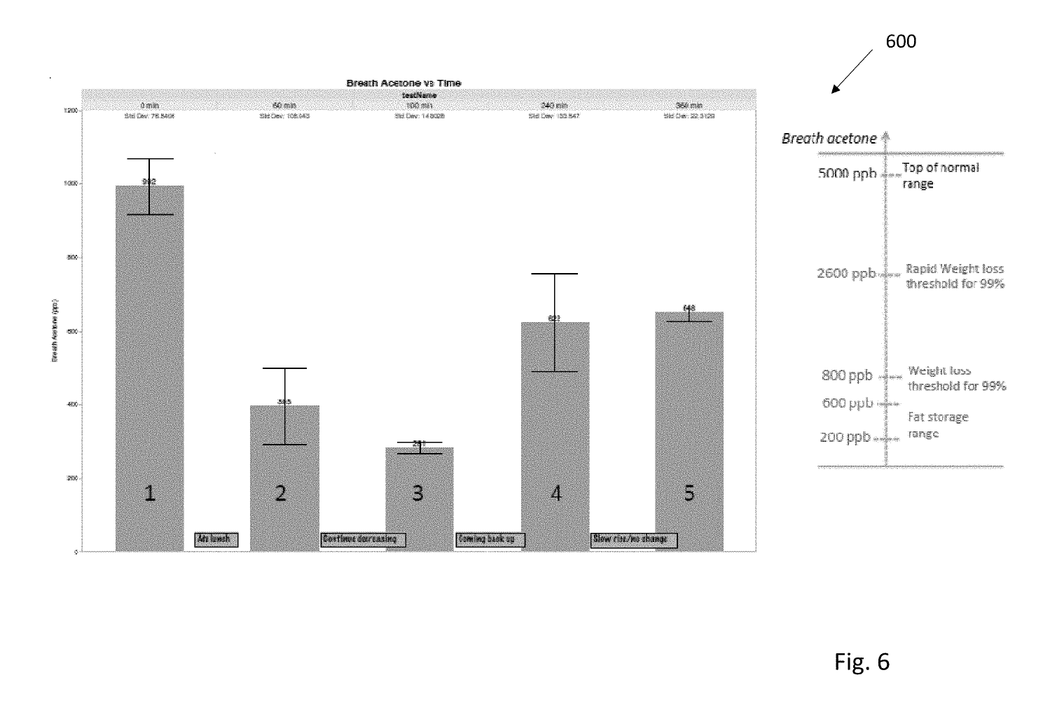

a. FIG. 6 is a graph 600 illustrating representative acetone readings and their metabolic significance for a typical user. As seen in the graph, three acetone measurements are taken and averaged for as indicated in the following:
1) User did not eat breakfast and had a 12 hour fast before first measurement
2) The user waited 30 minutes, finishing lunch to before re-measuring breath acetone, resulting in large decrease due to available carbohydrate
3) Acetone decreasing at 100 minutes
4) Metabolized much of available carbohydrate, acetone coming back up
5) Slow rise/no change at 360 minutes

As is apparent, breath acetone levels leading to weight loss and “fat burning” can be readily distinguished from levels at which fat is stored.

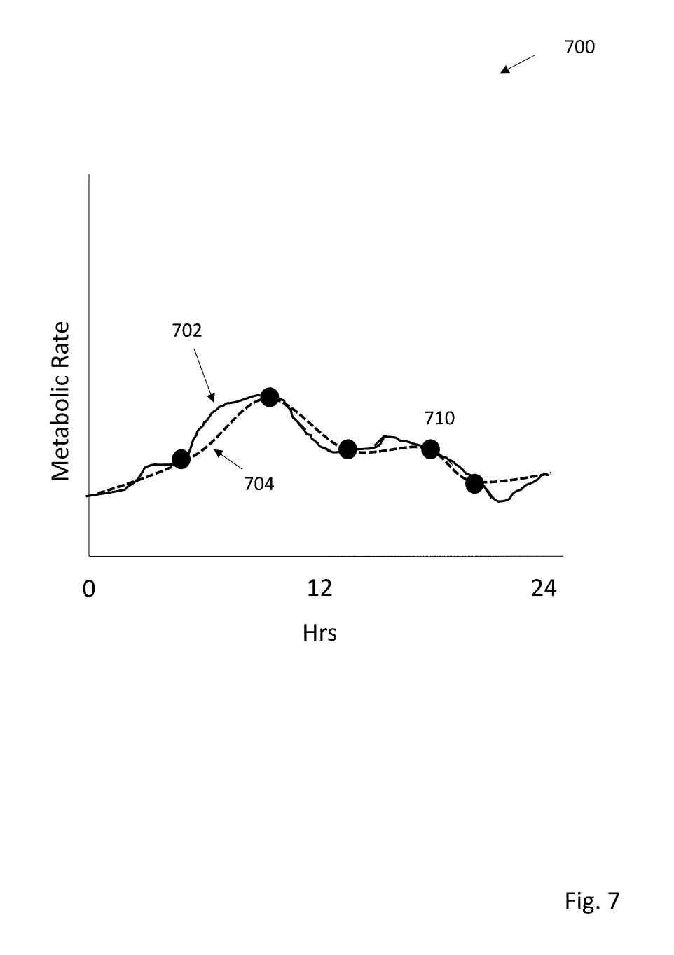

FIG. 7 is a graph 700 illustrating daily metabolic rate and five selected times (indicated by dots 710) when breath acetone measurements are completed. Actual metabolic rate is illustrated solid line 702, while estimated metabolic rate based on curve fitting through dots 710 is shown by dotted line 704. Total metabolic activity, and consequent fat burning activity, can be estimated by determination of area under dotted line 704. As will be understood, more frequent breath acetone measurements will allow for increased accuracy in metabolic rate estimates.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims. It is also understood that other embodiments of this invention may be practiced in the absence of an element/step not specifically disclosed herein.

What is claimed is:

1. A breath acetone measurement system comprising:
a multiple sensor array having
a first semiconductor sensor that measures concentration of a set of volatile organic compounds;
a second semiconductor sensor that measures concentration of a subset of the volatile organic compounds measured by the first semiconductor sensor;
a third electrochemical sensor;
at least one correction sensor that measures other breath properties; and
an acetone concentration calculation module that takes measured values from the multiple sensor array to measure breath acetone.

2. The breath acetone measurement system of claim 1, wherein the third electrochemical sensor measures at least one of a further subset of volatile organic compounds and hydrogen.

3. The breath acetone measurement system of claim 1, wherein at least one correction sensor is a water vapor sensor.

4. The breath acetone measurement system of claim 1, wherein at least one correction sensor is a carbon monoxide sensor.

5. The breath acetone measurement system of claim 1, wherein at least one correction sensor is a temperature sensor.

6. The breath acetone measurement system of claim 1, wherein at least one correction sensor is a pressure sensor.

7. A device for determining acetone contained in human breath, the device comprising:
a first semiconductor sensor that measures concentration of a set of volatile organic compounds in human breath;
a second semiconductor sensor that measures concentration of a first subset set of volatile organic compounds in the human breath, the first subset of volatile organic compounds being a subset of the set of volatile organic compounds;
an electrochemical sensor;
at least one correction sensor that measures one or more other breath properties of the human breath; and
an acetone concentration calculation module that determines the acetone contained in the human breath based on the concentrations of volatile organic compounds measured in each of: the set, the first subset, the second subset and the one or more other breath properties.

8. A breath acetone measurement system comprising:
a multiple sensor array having
a semiconductor sensor that measures concentration of a set of volatile organic compounds;
a electrochemical sensor;
at least one correction sensor that measures other breath properties;
an acetone concentration calculation module that takes measured values from the multiple sensor array to measure breath acetone; and
a smartphone supporting the acetone concentration calculation module and providing a visual display of results.

9. The breath acetone measurement system of claim 8, wherein the third electrochemical sensor detects ethylene oxide.

10. The breath acetone measurement system of claim 8, wherein at least one correction sensor is a water vapor sensor.

11. The breath acetone measurement system of claim 8, wherein at least one correction sensor is a carbon monoxide sensor.

12. The breath acetone measurement system of claim 8, wherein at least one correction sensor is a temperature sensor.

13. The breath acetone measurement system of claim 8, wherein at least one correction sensor is a pressure sensor.

14. A method for determining breath acetone, comprising the steps of:
providing a multiple sensor array for
measuring concentration of a set of volatile organic compounds using a first semiconductor sensor;
measuring concentration of a set of volatile organic compounds using a second semiconductor sensor that measures concentration of a subset of the volatile organic compounds measured by the first semiconductor sensor; and
measuring at least one of a further subset of volatile organic compounds and hydrogen using a third electrochemical sensor;
measuring other breath properties using at least one correction sensor; and
calculating breath acetone concentration using measured values from the multiple sensor array.

15. The breath acetone measurement system of claim 14, wherein the third electrochemical sensor detects ethylene oxide.

16. The breath acetone measurement system of claim 14, wherein at least one correction sensor is a water vapor sensor.

17. The breath acetone measurement system of claim 14, wherein at least one correction sensor is a carbon monoxide sensor.

18. The breath acetone measurement system of claim 14, wherein at least one correction sensor is a temperature sensor.

19. The breath acetone measurement system of claim 14, wherein at least one correction sensor is a pressure sensor.
